(19) United States
(12) Patent Application Publication
(10) Pub. No.: US 2002/0041900 A1
(43) Pub. Date: Apr. 11, 2002

(54) Chondrocyte cultures and fractions therefrom

(76) Inventors: Ole Olsen, Charlottenlund (DK); Morten Albrechtsen, Charlottenlund (DK); Marie-Louise Orbech Little, Horsholm (DK)

Correspondence Address: JACOBSON HOLMAN, PROFESSIONAL LIMITED LIABILITY COMPANY, 400 SEVENTH STREET, N.W., WASHINGTON, DC, 20004, US

(21) Appl. No.: 09/845,831

(22) Filed: May 1, 2001

(30) Foreign Application Priority Data
May 1, 2000 (DK) PA 2000 00712

(51) Int. Cl. A61K 35/64; C12N 5/06

(52) U.S. Cl. 424/538; 435/325

(57) ABSTRACT

The present invention concerns the development of anti-angiogenic, anti-inflammatoric, lysozomic and/or anti-collagenolytic agents and/or collagen and/or chondroitinsulphate from cultured chondrocytes. These chondrocytes are collected from a wide variety of animals, preferably elasmobranches cartilage and a detailed culturing procedure is described. The cartilage may be obtained from dead animals or as a biopsy from living animals. Then the chondrocytes are denuded from the extracellular matrix. Thereafter the chondrocytes are cultured to produce the agents, which are then isolated and purified.

### FIELD OF THE INVENTION

The present invention relates to a method of producing an anti-angiogenic, anti-inflammatoric, lysozomic and/or anti-collagenolytic fraction and/or collagen and/or chondroitinsulphate, in particular an anti-angiogenic fraction from cultured chondrocytes, in particular from cultured elasmobranch chondrocytes, an anti-angiogenic fraction, a method of culturing elasmobranch chondrocytes as well as a method of treating diseases with an anti-angiogenic, anti-inflammatoric, lysozomic and/or anti-collagenolytic fraction and/or collagen and/or chondroitinsulphate, in particular an anti-angiogenic fraction.

### BACKGROUND OF THE INVENTION

Cartilage is an avascularized tissue found in small quantities in humans and all most every creature, except sharks, rays, and other elasmobranches (cartilaginous fishes) where the entire skeleton is composed of cartilage. Cartilage has been studied as a potential candidate for containing anti-angiogenic factors. It is also a tissue that is relatively resistant to tumor development. The tumor associated with cartilage, chondrosarcoma, is the least vascularized of solid tumors. Angiogenesis is one of the important factors in the development of a tumor. Discrete solid tumoral masses appear if the tumor cells can provoke the adjacent vascular network to expand to supply their nutritional needs Therefore, the factors involved in the stimulation of angiogenesis have been studied for their role in the development of tumor and anti-angiogenic factors as well as drugs having an angiogenic inhibitory activity have also been investigated as tools for controlling the growth or for effecting regression of tumors.

Anti-angiogenic factors are not just investigated in elasmobranchs but the first initial studies were made using cartilage from calves. Here it was discovered that scapular cartilage in calves contains a substance that inhibits the vascularization of solid tumors (Langer et al., 1976). Because of its encouraging potential as anti-tumor agent, sources of greater supply of cartilage have been looked for.

Because of a very small content of cartilage in calves scientists were looking around for other creatures with a larger content of cartilage Therefore sharks became a potential source for this kind of angiogenesis inhibitor because their endoskeleton is composed entirely of cartilage (6% of their body weight as compared to 0.6% in calves). As an interesting characteristic sharks also have a low propensity to developing tumors. Many hypotheses have been elaborated to explain this low probability of developing tumors in sharks. Marchalonis et al. (1990) have shown IgM antibodies able to readily attack any aggressing agent. McKinney et al (1990) has shown that sharks have macrophages capable of differentiating normal cells from neoplastic cells and of destroying the latter. Rosen and Woodhead (1980) have postulated that the rarity of tumors in elasmobranchs might be due to the high ionic strength of their tissues, which is equivalent to a high body temperature. In these conditions, these authors believe that the immune system exerts a close to 100% immunological surveillance. Moore et al (1993) have discovered that sharks produce an aminosterol having antibacterial and antiprotozoal properties. Finally, Lee and Langer (1983) and Folkman and Klagsbrun (1987) have shown that sharks produce a substance that inhibits neovascularization. Lee and Langer (1983) have isolated this substance by extracting it from shark cartilage in denaturing conditions (guanidine extraction). This process of extraction is however very long (41 days) and might generate extracts having denatured factors. While the active substance isolated from calves has a molecular weight of about 16 kDa, the same group of researchers has not given a precise molecular weight to the one retrieved in shark. This substance is only defined has having a molecular weight higher than 3-5 kDa. Oikawa et al. (1990) have applied the same method of extraction as the one described by Lee and Langer, but of a much shorter duration (2 days instead of 41 days). The substance isolated from shark cartilage by Oikawa et al. has a molecular weight ranging from 1 to 10 kDa. Schinitsky (U.S. Pat. No. 4,473,551) has described a water extract of crude powdered shark cartilage in which a fraction of more than 100 kDa has an anti-inflammatory activity alone or in combination with glucosamine. No suggestion of a component of this extract having an anti-angiogenic or anti-tumor activity is made in this patent Kuetner et al. (U.S. Pat. No. 4,746,729) have isolated a polymorphonuclear neutrophil (PMN) elastase inhibitor from bovine cartilage. This inhibitor has been obtained from an aqueous extract of cartilage from which molecules of a molecular weight of more than 50 kDa have been retained. Fractionation on Sephacryl S-200 has given numerous fractions from which those of 10-40 kDa have been pooled after they have demonstrated an anti-elastase activity. The active component has an isoelectric point of 9.5 and might have a molecular weight of about 15 kDa. Kuetner et al. (U.S. Pat. No. 4,042,457) have also shown that bovine cartilage has a component of a molecular weight of less than 50 kDa which has a cell proliferation inhibitory activity without any activity on endothelial cell growth. Spilburg et al. (U.S. Pat. No. 4,243,582) have isolated two glycoproteins of molecular weight of 65 KDa and of pl 3.8 from bovine cartilage (guanidine-extraction) which show anti-trypsin activity and an endothelial cell growth inhibitory activity.

Dupont et al. has developed a shark cartilage extract which is extracted in water and gives two fractions (a pellet and a supernatant), the supernatant is further concentrated so they produced an extract with the ability to inhibit the angiogenesis, the extract is named AE-941 (or AE-941/Neovastat). Further characterisation of the extract showed that a single compound did not cause the anti-angiogenic activity but there was five different fractions with molecular weights 1-2.5 kDa; 29 kDa; 35-46 kDa; 60-70 kDa and 70-120 kDa all contributing to the anti-angiogenic activity (U.S. Pat. No. 5,618,925 and U.S. Pat. No. 5,985,835). This AE-941 extract was shown to inhibit endothelial cell proliferation in vitro, it has also shown an important inhibition against matrix metalloproteinases MMP-2 and MMP-12 and resent findings indicate that AE-941 also compete against the binding of vascular endothelium growth factor (VEGF) to its receptor in endothelial cells. Aeterna Laboratories, a Canadian biotechnology company and the inventor of this extract, has in May 1999 made explorations where cases in lung tumors; prostate tumors; breast tumors; refractory solid tumors; psoriasis and age-related macular degeneration has been conducted in Canada and USA. These explorations was as phase I/II trials where the purpose of the studies was to determine the optimal dose for an upcoming phase III trial and to explore the safety and tolerability of the extract. Very few cases of adverse events were found most of those reported were found in the gastrointestinal system and caused nausea and vomiting but non were assumed to be related to AE-941. It is then possible to conclude that AE-941 is an oral inhibitor of angiogenesis with an excellent safety profile for long-term administration alone or in combination with standard therapy. At present time a phase III trial has begun, this trial is sponsored by the National Cancer Institute (NCI) and is testing the AE-941 extract for the medical effect on cancer and psoriasis (No Author, 1999).

Cartilage consists essentially of highly specialised cells known as chondrocytes, surrounded by a dense extracellular matrix. In the case of articular cartilage, the issue is formed primarily from type II collagen, proteoglycans, and water.

Culturing of cartilage cells containing significant amounts of the anti-angiogenic factor is complicated by the inherent nature of cartilage tissue. Cartilage cells in vivo produce an extracellular matrix that separates one cell from another One of the main functions of the cells comprising cartilage would appear to be the production of the extracellular matrix. These cartilage forming calls are called chondroblasts. During the growth of an animal, the cartilage is growing and many cartilage cells retain their ability to reproduce.

In an adult animal, the majority of cartilage cells is embedded within the extracellular matrix and is non-reproductive and responsible for the maintenance of the tissue integrity. Even though functionally incorrect, all cartilage cells, whether reproductively active or inactive, are generally called chondrocytes. The extracellular matrix, consisting largely of collagen type II and proteoglycans, is extremely long lasting; cartilage collagen in certain animals may have a half-life of several years. Accordingly, when the chondrocytes have produced the extracellular matrix around themselves and thereby produced the cartilage tissue, they have generally served their function. Chondrocytes from adult cartilage tissue have generally lost their in vivo potential to reproduce.

The most actively reproducing chondrocytes are those found in embryonic tissue. However, embryonic tissue appears to contain less anti-angiogenic factor than adolescent tissue. Illustrative of this fact is that while adult cartilage tissue is nonvascular, as a result of the anti-angiogenic factor preventing the ingrowth of capillary cells, some early embryonic cartilage is supplied by the vascular system.

Chondrocytes, like any other connective tissue types, are both physically and chemically controlled by their environment. When culturing these cells under appropriate culture conditions, the cartilage producing cells will proliferate and maintain their differentiated character, i.e. secrete mainly collagen type II, the type that is characteristic of chondrocytes.

When the chondrocytes or cartilage cells are obtained from most cartilage tissue and cultured at conditions that do not favour the chondrocyte phenotype, a transformation or differentiation occurs of the cells. After a couple of days “incubation time” the chondrocytes, loose their typical rounded shape, flatten down on the substratum and stop secreting the cartilage matrix. The chondrocytes thus change their phenotypic behaviour and appears fibroblastic and starts to secrete collagen type I, the type that is characteristic of fibroblasts.

It has previously been shown for cells expanded in serum-containing medium that the differentiated state can be de-differentiated or reversed by transfering cells to a suspension culture in the presence of serum. A procedure used for testing the re-differentiation potential is to suspend these differentiated cells into agarose and staining for the typical components of the chondrocyte matrix (U.S. Pat. No. 6,150,1639).

In order to produce true cartilage tissue, therefore, it is necessary that the chondrocytes maintain or retain their differentiated state and proliferate only into cartilage type cells. With this, the culture of cartilage cells at conditions that favour the chondrocyte phenotype in such a way that these cells secrete cartilage components, for example, collagen type II, chondroitin sulphate and cartilage specific proteoglycans. The secretion of either of the cartilage extracellular matrix components, would indicate the chondrocyte phenotype and also the secretion of biological active components i.e. angiogenic, inflammatory and/or collagenolytic inhibitors.

It has been recognised that the relative amounts of collagen type I and collagen type II which are obtained from chondrocyte cultures depend on the environment surrounding the cultured chondrocytes. Different cellular environments give rise to cultures with collagen type I and collagen type II in varying amounts. Coon and Cahn (1966) described a technique for the cultivation of cartilage synthesising cells from chick embryo somites. Later Cahn and Lasher (1967) used the system for analysis of the involvement of DNA synthesis as a prerequisite for cartilage differentiation. Chondrocytes respond to both EGF and FGF by growth (Gospodarowicz and Mescher, 1977), but ultimately lose their differentiated function (Benya et al., 1978). Additionally, Kolettas at al. examined the expression of cartilage-specific molecules such as collagens and proteoglycans under prolonged cell culturing. They found that despite morphological changes during culturing in monolayer cultures (Aulthouse, A. et al., 1989; Archer; C. et al., 1990; Haanselmann, H. et al., 1994, Bonaventure, J. et al., 1994) when compared to suspension cultures grown over agarose gels, alginate beads or as spinner cultures (retaining a round cell morphology) the expressed markers such as types II and IX collagens and the large aggregating proteoglycans, aggrecan, versican and link protein did not change.(Kolettas, E. et al., 1995). Then methods for growing human chondrocytes were described by Brittberg, M. et al, 1994.

Then on the behalf of animal protection shark cartilage extracts could be very dangerous due to e.g. shark extension. Therefore alternative methods are desirable for achieving e.g. shark cartilage for the production of extracts usable for the treatment of angiogenic dependent diseases.

Although chondrocytes from a variety of animals and human beings have been cultured it has not been possible to obtain an anti-angiogenic fraction from these cultures. In particular the culturing of shark chondrocytes have not been reported.

One problem in culturing elasmobranches, such as sharks, are that they differ from human cell cultures and cannot be cultured by conditions proven to function when culturing for example human chondrocytes in relation to production of implants of cartilage.

As hereinabove described, the most active cartilage tissue, i.e. embryonic tissue, is limited in the production of anti-invasion factor, and mature cartilage is metabolically slow and contains cells which are generally inactive. Therefore, when mature tissue is degraded by the best known methods a very low yield of viable chondrocytes is provided. It is therefore desirable to obtain cells from non-embryonic animals that nevertheless provide a high percentage of viable chondrocytes.

### SUMMARY OF THE INVENTION

The present invention relates to a method of producing an anti-angiogenic, anti-inflammatoric, lysozomic and/or anti-collagenolytic fraction and/or collagen and/or chondroitinsulphate from cultured chondrocytes, preferably producing an anti-angiogenic, anti-inflammatoric, lysozomic and/or anti-collagenolytic fraction, comprising selecting a chondrocyte source having a sufficient amount of viable chondrocytes, denuding the chondrocytes from extracellular matrix, plating the denuded chondrocytes in high density in a culture medium, separating the cultured chondrocytes from the culture medium, extracting a fraction, comprising anti-angiogenic, anti-inflammatoric, lysozomic and/or anti-collagenolytic molecules and/or collagen and/or chondroitinsulphate from said cultured chondrocytes, obtaining the fraction. In particular the method relates to the production of an anti-angiogenic fraction. By the term “from said cultured chondrocytes” is meant that the fractions may be obtained from fractioning the cells, or the culture media and/or both depending on whether intracellular or extracellular fractions are desired.

Furthermore, in a preferred embodiment the method according to the invention uses elasmobranch chondrocyte sources obtaining an anti-angiogenic, anti-inflammatoric, lysozomic and/or anti-collagenolytic fraction and/or collagen and/or chondroitinsulphate, in particular an anti-angiogenic fraction.

By the term high density is meant that the density of chondrocytes is sufficient to allow the chondrocytes to grow and produce extracellular matrix.

Furthermore, the invention relates to a method of culturing elasmobranch chondrocytes comprising, selecting a chondrocyte source having a sufficient amount of viable chondrocytes, denuding the chondrocytes from extracellular matrix, plating the denuded chondrocytes in high density in a culture medium, and culturing the chondrocytes at a temperature below 37° C., such as below 29° C.

In another aspect the invention relates to an anti-angiogenic, anti-inflammatoric, lysozomic and/or anti-collagenolytic fraction, in particular an anti-angiogenic fraction obtainable by separating the cultured chondrocytes from the culture medium, and extracting a fraction, comprising anti-angiogenic molecules from said cultured chondrocytes, obtaining the anti-angiogenic fraction. The fraction is preferably a fraction obtained from elasmobranch chondrocytes. The fraction obtainable from said method is characterised in that it comprises substantially no molecules from contaminating cells, such as substantially no molecules from fat tissue, substantially no molecules from muscle tissue, and substantially no molecules from bone tissue. Furthermore, the fractions may comprise more active molecules as compared to the conventional fractions obtained from cartilage, thus it is possible to standardise a product comprising the fraction more easily. Also, the fractions according to the invention may be obtained without killing a corresponding amount of animals, in particular sharks, for example by obtaining a biopsy from the living shark, such as a biopsy from the fin.

Also, the invention relates to an anti-angiogenic, anti-inflammatoric, lysozomic and/or anti-colagenclytic composition, in particular an anti-angiogenic composition comprising one or more of the fractions defined above of cultured chondrocytes.

In yet another aspect the invention relates to a method of treating a disease of an animal or a human being comprising administering to the animal or human being a sufficient amount of one or more fractions as obtainable by the method according to the invention or a composition as defined in this invention.

Yet a further aspect of the invention is a chondrocyte culture as obtainable by selecting a chondrocyte source having a sufficient amount of viable chondrocytes, denuding the chondrocytes from extracellular matrix, plating the denuded chondrocytes in high density in a culture medium, and culturing the chondrocytes at a temperature below 29° C., in particular said chondrocyte culture relates to elasmobranch chondrocytes, such as shark chondrocytes.

### DETAILED DESCRIPTION OF THE INVENTION

By the term an anti-angiogenic fraction is meant a fraction having an anti-angiogenic activity as tested for example by the in vitro assay on hormone-dependent cell lines discussed below, wherein the anti-angiogenic activity is determined as the inhibition of growth of blood vessels observed in vivo in experimentally induced tumors. The anti-angiogenic fraction comprises cartilage molecules extracted from the chondrocyte culture.

The size of the cartilage molecules is preferably in the range between 0.5 kDa and 500 kDa, such as below 300 kDa.

More preferably the cartilage molecules have a size below 250 kDa. In a preferred embodiment the molecules are within the ranges of from 2-65, such as from 25-60, preferably from 30-50, and/or the ranges of from 70-120, such as from 80-100, preferably from 85-95, and/or the ranges of from 170-220, such as from 185-200.

The isoelectric point of the molecules of the anti-angiogenic fraction is normally in the range of between 9.0 and 10.0, more preferably in the range of between 9.2 and 9.7.

In general both water-soluble molecules as well non-water soluble molecules are fractionised. In particular with respect to the molecules in the lower ranges, the fractions comprising water-soluble molecules are selected.

The chondrocyte source may be any source of chondrocytes and from which chondrocytes being sufficiently viable to be cultured may be isolated. The chondrocyte source may thus be cartilage, in particular fresh cartilage. The chondrocyte source is preferably cartilage from animals known to produce anti-angiogenic substances in sufficient amounts. Also the cartilage may be selected from specific locations on the animal, such as corresponding to the scapula. By the term “chondrocytes” is meant any cell capable of being having a chondrocytes phenotype and/or capable of being converted into such a cell. Accordingly, the chondrocytes include immature as well as mature cells, such as stem cells, in particular embryonic stem cells, mesenchymal stem cells, chondroblasts, and chondrocytes.

In another embodiment the chondrocyte source may be a chondrocyte culture, optionally a chondrocyte cell line or immortalized chondrocytes capable of producing at least one of the molecules of interest.

Independent on type of chondrocyte source the chondrocytes may be from any species having cartilage with anti-angiogenic molecules. The chondrocytes may be from mammals, such as humans and calves. In particular the chondrocytes are elasmobranch chondrocytes, such as shark chondrocytes or ray chondrocytes. The shark or ray may be from any species, such as the Black Spiny Dog Fish, the Common Spiny Dog Fish (Squalus acanthias) the Japanese shark (Squatina californica), the Basking shark (Cetorhinus maximus), the Blue shark (Prionace glauca), and the Thresher (Alopias vulpinus).

Furthermore, the chondrocyte source may be any elasmobranch chondrocytes source having an anti-angiogenic, anti-inflammatoric, lysozomic and/or anti-collagenolytic molecules.

The shark chondrocyte source may in principle be any part of the shark cartilage, the present inventors have, however, shown, that it is advantageously to select specific parts of the cartilage skeleton, such as cranial cartilage since the growth rate of the cartilage exceed the growth rate of spinal cartilage for example. Other preferred parts are the jaws, and/or the fin. In particular when obtaining the chondrocytes by means of a biopsy, the fin is preferred.

In order to obtain an amount of anti-angiogenic molecules within a reasonable culturing period, the amount of viable chondrocytes, i.e. chondrocytes being capable of growing in the culture medium, is preferably at least 50%, such as at least 60%.

The viability of the chondrocytes may be assayed in conventional tests for viability, such as by staining the cells with Trypan Blue followed by counting the viable cells.

To promote the phenotypic behaviour of the chondrocytes, ie. produce and/or secrete the molecules of interest, specific environments have to be created. Factors such as enzymes used to isolate cells, temperature, oxygen level, pressure, cell seeding density and culture medium are all vital for the successful culture of these cells. Since successful culturing of elasmobranch chondrocytes has not previously been reported the present inventors have invented the culturing conditions for the elasmobranch chondrocytes.

Independent on the origin of the chondrocyte source, it is important that the chondrocytes are at least partly denuded before culturing, such as denuding the cells from substantially all extracellular matrix. By the term denuding is meant that the chondrocytes are isolated from the extracellular matrix so that substantially no contamination of the culture medium with extra-cellular foreign matrix is at risk. Thereby more pure fractions are obtained as compared to fractions obtained directly from cartilage. Denuding of the chondrocyte cells may be done by mechanically separating flesh from cartilage and subsequently enzymatically removing the remaining extracellular matrix and tighten up the cells. In another embodiment the denuding is at least partially, in that the culturing is conducted as explant cultures, that is, very thin slices of cartilage are plated in thin culture dishes to allow for chondrocyte outgrowth.

The denuded or isolated cells are suspended and/or plated in the culture medium in high density as discussed above.

In order to allow the chondrocytes to produce a sufficient amount of anti-angiogenic, anti-inflammatoric, lysozomic and/or anti-collagenolytic molecules it is important the plating is carried out so that the density is homogenous throughout the culture medium.

In order to test whether the density is sufficient for production of anti-angiogenic molecules it is sufficient to determine the percentage of collagen type II as compared to collagen type I produced. The higher the percentage the better conditions for producing anti-angiogenic molecules.

The plating or suspension density is preferably at least 1×10⁵ chondrocytes per ml, such as at least 2×10⁵ chondrocytes per ml, such as at least 3×10⁵ chondrocytes per ml, such as at least 4×10⁵ chondrocytes per ml, such as at least 5×10⁵ chondrocytes per ml, such as at least 6×10⁵ or chondrocytes per ml, such as at least 7×10⁵ chondrocytes per ml, such as at least 8×10⁵ chondrocytes per ml, such as at least 9×10⁵ chondrocytes per ml, such as at least 1×100 chondrocytes per ml.

The chondrocytes may be cultured in a monolayered culture, it is, however preferred to mimic the natural environment and culture the chondrocytes in a three-dimensional culture, such as culturing at a scaffold or a mould with or without surface modification. Such a scaffold or mould is preferably degradable within the biological environments, however this is no prerequisite, and the scaffold or mould may be non-degradable within the biological environments. Thus, the scaffold or mould may be composed of polymeric, ceramic or metallic substances, and/or the scaffold or mould include substances of natural or synthetic materials such as catgut and marine products, such as including substances of extra-cellular matrix components of allograft, autograft or xenograft origin or any combinations of these or other products produced in cultures such as collagen, proteoglycans and glycosaminglycans. The scaffold or mould may also include gels such as collagen gels and agarose gels. In yet another embodiment the chondrocytes are cultured in a bioreactor.

Furthermore, mechanical stress may be applied to the culture, such as shear stress, said stress preferably being constant during culturing.

Accordingly, the biochemical, physical and structural environments is approximately as in natural cartilage, and is preferably kept constant during culturing.

The chondrocytes may be cultured in any suitable media, such as medias obtained from Life Technologies. These media may be Medium 199, RPMI 1640, Nut.Mix.F12, DMEM/F12, DMEM high glucose. Also Schneider's insect media and Grace's insect media preferably modified to produce the fractions of interest. For culturing elasmobranch chondrocytes, in particular shark chondrocytes; the media used are preferably adjusted with respect to osmolarity, to obtain an osmolarity mimicking the naturally occurring osmolarity. Thus, the osmolarity is preferably in the range of from 600 to 1200 mosmol⁻¹, such as preferably in the range of from 800 to 1100 mosmol⁻¹, such as preferably in the range of from 900 to 1100 mosmol⁻¹.

The osmolarity may be adjusted by adding salt(s) to the media as well as adding urea. Elasmobranch retain urea in order to prevent osmotic water ion in the saline environment. Thus, urea is also added to mimic the naturally occurring environments for chondrocytes in sharks and rays.

It is preferred that the salt (NaCl) concentration of the media is from 0.14-1.00 M, such as from 0.20-0.30 M. Thus, in particular the media mentioned above may be modified by increasing the salt concentration to the ranges specified. In particular in relation to elasmobranch chondrocytes the medium may contain trimethylamine oxide (TMO).

The chondrocytes are cultured under aseptic conditions, and preferably antibiotics, such as penicillin, streptomycin, and fungizone, are added to the culture medium. Contamination of other cell types than chondrocytes may be eliminated during the culturing and isolation methods by any suitable method known to the person skilled in the art, such as by labelling and separation. Also the other cells may be eliminated by prolonging the culturing and/or isolation period up to 5 days, such as 6 days, such as 7 days, whereby other cell types have died, and the viable cells present are substantially only chondrocytes.

The culturing pressure is preferably in the range of from 1 to 10 atm, such as in the range of from 2 to 8 atm., such as in the range of from 2 to 5 atm. The oxygen level is normally selected about 5%, however the oxygen level may be in the range of from 1% to 20%, such as in the range of from 5% to 20%. The pressure is preferably constant during culturing.

The medium is preferably containing hormones and/or other growth factors known to stimulate certain behavior of chondrocytes, such as transforming growth factor-b, fibroblast growth factor and or insulin-like growth factor. Furthermore, the medium may contain serum and/or serum substitutions. Also, the medium may contain extracellular matrix (ECM) proteins, such as vitronecrion, fibronectin.

Independent of the media, it is preferred that a constant flow of nutrition toward the cells is obtained, and even more preferred that a constant flow of waste from the cultured cells is obtained.

In order to facilitate the cell culturing and obtain a high yield of anti-angiogenic molecules the culturing temperature is lower than usually applied during cell culturing, such as preferably below 29° C., such as preferably below 21° C., more preferably below 15° C.

In particular for elasmobranch chondrocytes it has been shown by the present inventors, that the temperature optimum may be below 15° C., such as about 8-10° C.

To allow the chondrocytes to settle in the culture medium, the chondrocytes are incubated at least 24 hours before changing culture medium. The longer settling period the better; and this must be balanced against the risk of having increased pH in the culture medium with time. Normally it is preferred to allow for 2 settling period of at least 48 hours, such as at least 36 hours. Thereafter the culture medium may be changed as is conventional practice, depending on the size of the culture plate.

The length of the incubation period is preferably enough to allow at least 10 cell division cycles, such as at least 25 cell division cycles, more preferred 50 cell division cycles, most preferred at least 100 cell division cycles.

Furthermore, the chondrocyte source may comprise chondrocytes being genetically manipulated, such as immortalised chondrocytes, or chondrocytes manipulated in other ways than genetic, such as by addition of hormones, other growth factors or physically manipulated, as discussed above.

When the growth period is finished after a sufficient number of cell division cycles the chondrocytes are separated from the culture media, by conventional techniques such as centrifugation. The culture media is preferably stored for further treatment, such as fractioning, since this medium may contain molecules of interest, and may therefore be used as such or added to one or more of the other fractions obtained, before further purification.

The isolation of the anti-angiogenic, anti-inflammatoric, iysozomic and/or anti-collagenolytic, in particular anti-angiogenic components can be performed according to a variety of procedures, including many of the fraction methods known to fraction cartilage as such, vide for example U.S. Pat. No. 5,075,112, U.S. Pat. No. 4,243,582, U.S. Pat. No. 3,478,146 and U.S. Pat. No. 4,350,682, as well as references mentioned in the introductory part of this description, as examples of how to obtain fractions from cartilage. These methods are typically classifiable as either mechanical/physical fractioning or chemical/enzymatic fractioning or combinations. In particular at least two different procedures are preferred, these procedures capable of being used separately or support one another to achieve the best and highest concentration of components of interest.

The first procedure is performed in particular to obtain the small molecules and is an extraction performed in water to get hold of the water-soluble molecules, in particular the small molecules. A homogenate from the chondrocytes, disrupted to release any anti-angiogenic molecules from the cytoplasma and separated from culture media, is obtained, and then extraction is performed on the homogenate, for example such as described in Example 2. A separation procedure may then be conducted, such as a centrifugation procedure whereby the resulting supernatant comprises the water-soluble molecules. The supernatant may be used as such, or further purified, dried and/or combined with other fractions. The retentate may be discarded or subjected to a second extraction, such as the following second procedure.

The second procedure is performed to retrieve as much of the anti-angiogenic molecules not water-soluble as possible by using organic solvents, chactropic agents, detergents, or other kinds of additives in the extraction solution.

The two procedures may be conducted in combination, so that first the fractions containing water-soluble molecules are obtained and subsequently the other fractions are obtained. Alternatively, either of the procedures may be conducted only.

Thus the second extraction procedure could either be performed using cells/cartilage directly from the culture tank (without pre-water-extraction) or it could be performed using cells/cartilage from pre-water-extraction.

When isolating fraction comprising molecules not soluble in water the cells/cartilage could be extracted using a solvent such as guanidine hydrochloride. Often the solvent will be used in a concentration of about 1-6M solution.

After this extraction the solvent, such as guanidine hydrochloride has to be removed. Either dialysis or ultrafiltration using cut-off values of 5-10 kDa removes these additives.

Anti-angiogenic fractions are obtained from the culture media, the supernatant from the water-extraction as well as the non-water extraction. Also the retentate from the non-water-extraction could serve as a fraction.

The various fractions from two extraction procedures and the culture medic can be pooled to obtain combined fractions, wherefrom the further purification/concentration/analysis can be made.

The fractions obtained may be further concentrated by any suitable means, such as by ultra-filtration. In order to obtain a fraction comprising the predetermined molecules, the ultrafiltration may be carried out with an filter membrane having a porosity of about 1500 kDa, such as about 500 kDa, preferably about 250 kDa.

Further purification of the fraction may be carried out by flocculation, precipitation and/or chromatography.

For chromatography the column used can be packed bed adsorption, expanded bed adsorption or magnetic separation. The separation techniques used can be gel filtration, ion exchange, hydrophobic interactions, reversed phase, metal chelate, affinity, and covalent bindings.

The anti-angiogenic fraction is suitably capable of being stored for a longer period before use. It is preferred to dry the fraction, such as by spray-drying or freeze-drying to obtain a powder having superior storage properties compared to liquid preparations.

Chondroiting sulphate and collagen may be obtained from the cell cultures using either physical or chemical separation and purification techniques. For example, collagen may be obtained by centrifuging the cell culture medium and cells at 150 g/1,000 r.p.m. for 10 minutes, so to obtain a pellet and supernatant. The pellet, containing the cell suspension is discarded and the collagen in the supernatant is centrifuged again at 600 g for 10 minutes. The pellet, containing insoluble collagen, is suspended in a buffer and desalted by a combination of, for example, heating treatment and enzyme digestions to thereby obtain a liquid collagen. This liquid collagen may be further purified to eliminate any kind of viruses.

Composition

The anti-angiogenic fraction obtained may be used as any conventional anti-angiogenic fraction obtained directly from cartilage. Due to the method according to the invention it is possible to obtain substantially pure fractions, that is substantially without any substances relating to cartilage as such or other connective tissue that is present in fractions obtained by homogenising and fractionating cartilage as such. Thereby contamination of foreign molecules in the composition is avoided. Furthermore, it is easier to standardise the fractions obtained for further purposes, such as for the preparation of pharmaceutical compositions.

One aspect of the invention relates to a composition comprising a fraction according to the invention, said composition being suitable for administering to animals and/or human beings, in particular said composition is a pharmaceutical composition.

The main routes of drug delivery, are intravenous, oral, and topical, as will be described below. Other drug-administration methods, such as subcutaneous injection or via inhalation, which are effective to deliver the drug to a target site or to introduce the drug into the bloodstream, are also contemplated.

The mucosal membrane to which the pharmaceutical preparation of the invention is administered may be any mucosal membrane of the mammal to which the biologically active substance is to be given, e.g. in the nose, vagina, eye, mouth, genital tract, lungs, gastrointestinal tract, or rectum, preferably the mucosa of the nose, mouth or vagina.

Fractions of the invention may be administered parenterally, that is by intravenous, intramuscular, subcutaneous intranasal, intrarectal, intravaginal or intraperitoneal administration. The subcutaneous and intramuscular forms of parenteral administration are generally preferred. Appropriate dosage forms for such administration may be prepared by conventional techniques. The fractions may also be administered by inhalation, that is by intranasal and oral inhalation administration. Appropriate dosage forms for such administration, such as an aerosol formulation or a metered dose inhaler, may be prepared by conventional techniques.

The fractions according to the invention may be administered with at least one other fraction, such as an anti-cancer fraction. The fractions may be administered simultaneously, either as separate formulations or combined in a unit dosage form, or administered sequentially.

The dosage requirements will vary with the particular drug composition employed, the route of administration and the particular subject being treated. Ideally, a patient to be treated by the present method will receive a pharmaceutically effective amount of the fraction in the maximum tolerated dose, generally no higher than that required before drug resistance develops.

For all methods of use disclosed herein for the fractions, the daily oral dosage regimen will preferably be from about 0.01 to about 80 mg/kg of total body weight. The daily parenteral dosage regimen about 0.001 to about 80 mg/kg of total body weight. The daily topical dosage regimen will preferably be from 0.1 mg to 150 mg, administered one to four, preferably two or three times daily. The daily inhalation dosage regimen will preferably be from about 0.01 mg/kg to about 1 mg/kg per day. It will also be recognized by one of skill in the art that the optimal quantity and spacing of individual dosages of a fraction or a pharmaceutically acceptable salt thereof, will be determined by the nature and extent of the condition being treated, the form, route and site of administration, and the particular patient being treated, and that such optimums can be determined by conventional techniques. It will also be appreciated by one of skill in the art that the optimal course of treatment, i.e., the number of doses of a fraction or a pharmaceutically acceptable salt thereof given per day for a defined number of days, can be ascertained by those skilled in the art using conventional course of treatment determination tests.

The term “unit dosage form” as used herein refers to physically discrete units suitable as unitary dosages for human and animal subjects, each unit containing a predetermined quantity of a fraction, alone or in combination with other agents, calculated in an amount sufficient to produce the desired effect in association with a pharmaceutically acceptable diluent, carrier, or vehicle. The specifications for the unit dosage forms of the present invention depend on the particular fraction or fractions employed and the effect to be achieved, as well as the pharmacodynamics associated with each fraction in the host. The dose administered should be an “effective amount” or an amount necessary to achieve an “effective lever” in the individual patient.

Since the “effective level” is used as the preferred endpoint for dosing, the actual dose and schedule can vary, depending on interindividual differences in pharmacokinetics, drug distribution, and metabolism. The “effective level” can be defined, for example, as the blood or tissue level desired in the patient that corresponds to a concentration of one or more fractions according to the invention.

Pharmaceutical compositions containing a fraction of the present invention may be prepared by conventional techniques, e.g. as described in Remington, The Science and Practice of Pharmacy 1995, edited by E. W. Martin, Mack Publishing Company, 19th edition, Easton, Pa. The compositions may appear in conventional forms, for example capsules, tablets, aerosols, solutions, suspensions or topical applications.

Whilst it is possible for the compounds or salts of the present invention to be administered as the raw fraction, for example freeze, it may be preferred to present them in the form of a pharmaceutical formulation. Accordingly, the present invention further provides a pharmaceutical formulation, for medicinal application, which comprises a fraction of the present invention or a pharmaceutically acceptable salt thereof, as herein defined, and a pharmaceutically acceptable carrier therefor.

The fractions of the present invention may be formulated in a wide variety of oral administration dosage forms. The pharmaceutical compositions and dosage forms may comprise the fractions of the invention or its pharmaceutically acceptable salt or a crystal form thereof as the active component. The pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, solubilizers, lubricants, suspending agents, binders, preservatives, wetting agents, tablet disintegrating agents, or an encapsulating material.

Preferably, the composition will be about 0.5% to 75% by weight of a fraction or fractions of the invention, with the remainder consisting of suitable pharmaceutical excipients. For oral administration, such excipients include pharmaceutical grades of mannitol, lactose, starch, magnesium stearate, sodium saccharine, talcum cellulose, glucose, gelatin, sucrose, magnesium carbonate, and the like.

In powders, the carrier is a finely divided solid which is a mixture with the finely divided active component. In tablets, the active component is mixed with the carrier having the necessary binding capacity in suitable proportions and compacted in the shape and size desired. The powders and tablets preferably containing from one to about seventy percent of the active fraction. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active fraction with encapsulating material as carrier providing a capsule in which the active component, with or without carriers, is surrounded by a carrier, which is in association with it. Similarly, cachets and lozenges are included. Tablets, powders, capsules, pills, cachets, and lozenges can be as solid forms suitable for oral administration.

Drops according to the present invention may comprise sterile or non-sterile aqueous or oil solutions or suspensions, and may be prepared by dissolving the active ingredient in a suitable aqueous solution, optionally including a bactericidal and/or fungicidal agent and/or any other suitable preservative, and optionally including a surface active agent. The resulting solution may then be clarified by filtration, transferred to a suitable container which is then sealed and sterilized by autoclaving or maintaining at 98-100 degree. C. for half an hour. Alternatively, the solution may be sterilized by filtration and transferred to the container aseptically. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Other forms suitable for oral administration include liquid form preparations including emulsions, syrups, elixirs, aqueous solutions, aqueous suspensions, toothpaste, gel dentrifrice, chewing gum, or solid form preparations which are intended to be converted shortly before use to liquid form preparations. Emulsions may be prepared in solutions in aqueous propylene glycol solutions or may contain emulsifying agents such as lecithin, sorbitan monooleate, or acacia. Aqueous solutions can be prepared by dissolving the active component in water and adding suitable colorants, flavors, stabilizing and thickening agents. Aqueous suspensions can be prepared by dispersing the finely divided active component in water with viscous material, such as natural or synthetic gums, resins, methylcellulose, sodium carboxymethylcellulose, and other well known suspending agents. Solid form preparations include solutions, suspensions, and emulsions, and may contain, in addition to the active component, colorants, flavors, stabilizers, buffers, artificial and natural sweeteners, dispersants, thickeners, solubilizing agents, and the like.

The fractions of the present invention may be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol. Examples of oily or nonaqueous carriers diluents, solvents or vehicles include propylene glycol, polyethylene glycol, vegetable oils (e.g.; olive oil), and injectable organic esters (e.g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilisation from solution for constitution before use with a suitable vehicle, e.g., sterile, pyrogen-free water.

The parenteral formulations typically will contain from about 0.5 to about 25% by weight of the fraction in solution. Preservatives and buffers may be used. In order to minimize or eliminate irritation at the site of injection, such compositions may contain one or more nonionic surfactants having a hydrophile-lipophile balance (HLB) of from about 12 to about 17. The quantity of surfactant in such formulations will typically range from about 5 to about 1.5% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The paranteral formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

The fractions of the present invention may be formulated for administration as suppositories. A low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

The active fraction may be formulated into a suppository comprising, for example, about 0.5% to about 50% of a fraction of the invention, disposed in a polyethylene glycol (PEG) carrier (e.g., PEG 1000 [96%] and PEG 4000 [4%].

The fractions of the present invention may be formulated for vaginal administration. Pessaries, tampons, creams, gels, pastes, foams or sprays containing in addition to the active ingredient such carriers as are known in the art to be appropriate.

The fractions of the present invention may be formulated for nasal administration. The solutions or suspensions are applied directly to the nasal cavity by conventional means for example with a dropper, pipette or spray. The formulations may be provided in a single or multidose form. In the latter case of a dropper or pipette this may be achieved by the patient administering an appropriate, predetermined volume of the solution or suspension. In the case of a spray this may be achieved for example by means of a metering atomizing spray pump.

The fractions of the present invention may be formulated for aerosol administration, particularly to the respiratory tract and including intranasal administration. The fraction will generally have a small particle size for example of the order of 5 microns or less. Such a particle size may be obtained by means known in the art, for example by micronization. The active ingredient is provided in a pressurized pack with a suitable propellant such as a chlorofluorocarbon (CFC) for example dichlorodifluoromethane, trichlorofluoromethane, or dichlorotetrafluoroethane, carbon dioxide or other suitable gas. The aerosol many conveniently also contain a surfactant such as lecithin. The dose of drug may be controlled by a metered valve. Alternatively the active ingredients may be provided in a form of a dry powder, for example a powder mix of the fraction in a suitable powder base such as lactose, starch, starch derivatives such as hydroxypropylmethyl cellulose and polyvinylpyrrolidine (PVP). The powder carrier will form a gel in the nasal cavity. The powder composition may be presented in unit dose form for example in capsules or cartridges of e.g., gelatin or blister packs from which the powder may be administered by means of an inhaler.

When desired, formulations can be prepared with enteric coatings adapted for sustained or controlled release administration of the active ingredient.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The fraction or composition according to the invention is particular useful for being used in a method of treating and/or preventing a disease in an animal or human being, comprising administering to a patient a sufficient amount of the fraction or the composition. In particular the anti-angiogenic fraction or composition is useful for treating disease wherein angiogenesis, i.e. growth of blood vessels, is desirable to avoid, such as in cancer.

Accordingly, the present invention in particular relates to the treatment and/prevention of the following diseases:

Any disease being related to angiogenesis, such as any cancer disease. In particular the disease may be selected from malignant neoplasms of the gastro-intestinal tract, malignant neoplasms of the uro-genital organs, sarcomes and carcinomas of bone, muscle, connective tissue, skin and breast, hepatomas, malignant neoplasms of the lung, leukaemia and other blood cell and lymphatic related neoplasms as well as other malignant diseases, such as those mentioned in the WHO Classification of Diseases and Injuries.

Also, any immunological disease may be treated and/or prevented, such as immunological diseases where immunological elements are dominating the pathogenesis and where an immunological element is central in the investigation, diagnosis, treatment or control of the disease. In immunological diseases the structure or function of the damage organ or tissue is caused by a reaction between antigen and antibody, between antigen and immune cells or due to a malfunction of the immune system. Examples of immunological disease are infection diseases, allergic diseases, autoimmune disease (e.g. rheumatoid arthritis), immune defects, or transplantation of organs.

Furthermore, any diabetes related disease, in particular in relation to angiogenesis, such as diabetic renopati or diabetic retinopati.

A wide variety of other diseases may be treated and/or prevented by the present invention, such as psoriasis and lupus.

Furthermore, the invention relates to a chondrocyte culture as obtainable by

selecting a chondrocyte source having a sufficient amount of viable chondrocytes.

denuding the chondrocytes from extracellular matrix

plating the denuded chondrocytes in high density in a culture medium

culturing the chondrocytes at a temperature below 29° C.

In particular the method relates to culturing of elasmobranch chondrocytes.

The chondrocyte culture obtained may be stored appropriately for later use, such as for fractioning or function as a chondrocyte bank for subsequent culturing or function immediately, as a chondrocyte source for the production of anti-angiogenic fractions as discussed above.

### EXAMPLES

In the following non-limiting examples of the invention are described.

### Example 1

Culturing of shark chondrocytes:

The species used was a young dogfish shark (Scyliorphinus canicula) approximately 3-5 years of age. The shark was kept at the Department of Biological Sciences at Plymouth University, England. And in this example a modification of DMEM/F12 was used.

For cultivations of chondrocytes of elasmobranches, various medias can be modified to resemble elasmobranch plasma and be used with great efficiency, for example, Medium 199, RPMI 1640, L15 Glutamax, Nut. Mix. F12, DMEM/F12, DMEM high glucose. These medias can all be obtained from either Sigma Aldrich or Life Technologies.

Materials used:

ITS+ Premix from Becton Dickinson, Catalogue No 354352, LOT No. 001330 Nu-Serum IV Culture Supplement from Becton Dickinson, Catalogue No. 355101, LOT No 003920.

Isolation of Cartilage

Chicken scissors, scalpels, tweezers and laminar flow cabinet are thoroughly cleaned using 70% ethanol, to avoid bacterial and fungous contamination.

Prior to the procedure, the shark is killed by an overdose of anaesthetics by placing this in a container with 821 mg 3-aminobenzoic acid ethyl ester (Sigma Aldrich) dissolved in 4 liters saline water. To ensure death, a hard stroke is placed on the back of the head of the shark using a 30 cm long iron bar, which has also been treated with 70% ethanol to avoid contamination.

All the following treatments from now until the end product are performed under aseptic conditions using sterile labcoats, hair and mouth protection and sterile gloves. During the preparation and dissection of the cartilage, it is considered important to keep the temperature relatively low, so to limit potential damage to the cells by room temperature.

After the shark has been killed, it is sprayed with 70% ethanol and placed on ice in a container in the laminar flow cabinet. A large area around where the initial incision is to be made, is sprayed with 70% ethanol. The spinal cord is transacted and an incision is made from the cranium along the spinal column using scalpel and chicken scissors. The edges of the skin and flesh around the cartilage are cleaned with 70% ethanol. The cartilage from the spinal cord, cranial, jaw, pelvic girdle and fin tissues are all cut out and cleaned as mush as possible to get rid of nervous tissue, adherent flesh and connective tissue. Keeping the different types of obtained cartilage apart, these are placed and washed in tissue culture dishes containing a sterile ice-cold Shark Ringer solution. This Shark Ringer solution is made from 1:1 Hank's Balanced salt solution (with sodium bicarbonate, without phenyl red; Sigma Aldrich) with the addition of 0.14M NaCl, 0.35M urea and penicillin (200 units/ml)/ streptomycin (200 μg/ml), so to mimic shark serum osmolarity. Small pieces of cartilage are placed in 10% formalin for future histological analysis.

The cartilage is minced into very fine pieces, approximately 1-2 mm² and are placed in glass bottles with 0.25% Trypsin/ Hank's Balanced salt solution (Sigma Aldrich) for 60 minutes or overnight at 9° C. on a magnetic stirrer at medium speed. The enzyme solution cover the cartilage pieces and works to remove adherent tissue. After the specific time period, the trypsin solution is removed and the cartilage is washed twice in the Shark Ringer solution (as described previously), however, with added 0.0098M CaCl₂ (anhydrous).

Cartilage is subjected to a series of digestion protocols using 0.25% collagenase type XI from Clostridium histolyticum (Sigma Aldrich) in the Shark Ringer solution with added CaCl₂ (so to fully activate the enzyme) Cartilage is placed in this solution at 9° C. overnight on a magnetic stirrer at medium speed. Cell release from this digestion is monitored initially after 12 hours and then every two hours.

At the completion of each digestion the enzyme-cell solution is filtered through a cell strainer (Becton Dickinson) into a 50 ml polypropylene Falcon Tube (Becton Dickinson), so to separate debris products from soluble molecules and cells (a new collagenase solution is added to the new digestion protocol).

To inhibit the enzymatic activity, cells are washed three times with Dulbecco's modified Eagle's-Ham's F-12 medium (1:1) containing 15 mM N-2-hydroxyethylpiperazine-N′-2-ethanesulfonic acid (HEPES) modified to resemble shark plasma by the addition of 100 mM NaCl, 3.9 mM CaCl₂, 2.5 mM MgCl₂, 300 mM urea, and 150 mM trimethylamine oxide. This nutrient medium is also supplemented with serum and/or serum supplements. In one experiment 10% Nu-Serum™ IV Culture Supplement, 1% ITS+™ Premix (Becton Dickinson), in another with 10% serum from the shark it-self. Furthermore, penicillin (200 units/ml)/ streptomycin (200 μg/ml) are added in both experiments. Each wash is followed by centrifuge for 10 minutes at 150 g at 9° C. to remove non-cellular components. Supernatants are discarded.

Cultivation of Chondrocytes

Still under aseptic conditions, the chondrocytes are resuspended in Dulbecco's modified Eagle's Ham's F-12 medium (as described above).

Cell viability of the suspensions is examined using a small amount of the cell suspension with Trypan Blue stain, the viability is then examined using a hemacytometer.

The cell suspensions are transferred to tissue culture dishes and cultured at high density of viable cells, at either 2×10⁵, 5×10⁵ or 1×10⁶ cells per ml to favour viability, differentiation and cell growth. The cultures are incubated at 15° C. with the addition of 5% CO₂. After an incubation time of 24 hours, cell culture medium is reseeded into a new culture dish so to allow for the attachment and growth of chondrocytes that are still in suspension The original cell culture is washed twice with new medium to remove unsettled debris, and the medium is then replaced. Depending on the type and size of the tissue culture dish, the medium is changed at regular intervals. For this experiment 24-well tissue culture plates are used. These wells contained 1 ml medium, and this medium is replaced every 2-3 days.

The culture media that is withdrawn from the cell cultures is placed in sterile containers at −20° C. for further examination.

After the initial 7 days, cell culture medium is added with only half the amount of penicillin (100 units/ml)/streptomycin (100 μg/ml), so to promote increased growth rate.

Examination of Phenotypic characteristics

At regular intervals cell cultures are fixed and stained with Giemsa for cell viability, aqueous toluidine blue for metachromasia (indicating the active synthesis of proteoglycans), Safranin O for sulphated glycosaminoglycans and Van Gieson's stain for collagen.

Isolation of the chondrocyte phenotype

In an attemp to isolate the chondrocytes, either the primary cell suspension obtained from the cartilage or a harvested monolayer (not necessarily confluent) from the tissue culture dish containing differentiated chondrocytes (appear fibroblastic and decrease and/or stop the synthesis of cartilage specific components), is placed in a liquid suspension culture. The harvested monolayer are obtained by removing cell culture medium from the culture dish, washing with phosphate buffered saline to remove serum components, adding a 0.25% trypsin solution (as described previously) until cells have detached, washing the suspension twice with cell culture medium, to stop the enzymatic reaction and centrifuge for 10 minutes at.

It was found that the chondrocytes and other cells were unable to plate out in the 0.5% agar and therefore remained in liquid suspension. Chondrocytes can live in suspension for at least 7 days, whereas other connective tissue cells such as the fibroblast, do not survive for more than 2-3 days.

The liquid suspension culture may be obtained by coating the bottom of a petridish with 0.5% agar (or agarose), a modification of the method explained by Klagsbrun (1979; “Large-Scale Preparation of Chondrocytes”, Methods in Enzyymology, Volume XXIII page 560-564). 1% solution of agar (or agarose) is made up in tissue culture water and sterilized by autoclaving, The solution is cooled to 45° C. and mixed with an equal amount of x2 Dulbecco's modified Eagle's-Ham's F-12 medium (1:1) containing 15 mM N-2-hydroxyethyl-piperazine-N′-2-ethanesulfonic acid (HEPES) modified to resemble shark plasma by the addition of 100 mM NaCl, 3.9 mM CaCl₂, 2.5 mM MgCl₂, 300 mM urea, and 150 mM trimethylamine oxide. This nutrient medium was also supplemented with 10% Nu-Serum™ IV Culture Supplement, 1% ITS+™ Premix (Becton Dickinson), penicillin (200 units/ml)/ streptomycin (200 μg/ml). Two mililiters of the 0.5% agar solution is poured into a 35 mm tissue culture dish. After the agar hardens, 3 ml of the cell suspension of a lower density of 2×10³ cells/per ml or the higher density for example 1×10⁸, is suspended or resususpended in the medium described above with serum supplements 10% Nu-Serum™ IV Culture Supplement, 1% ITS+™ Premix, and plated onto the agar plates.

After 3-4 days in this suspension, the medium is removed and reseeded onto tissue culture plates at desired density. Viability tests are carried out using a small amount of the cell suspension with Trypan Blue, these are examined in a hemacytometer.

Results:

Cultures are examined initially after 24 hours using an inverted microscope, and then every time culture medium is changed. In the cultures in which cells are seeded straight onto the tissue culture plate after being released from the cartilage matrix, a mixture of cells and debris can be seen. In the cultures that were initially seeded in liquid suspension, only the chondrocyte cells should have survived. In both type cultures, the chondrocytes can be seen as rounded shapes on their own or in dusters. After 3-5 days in culture, these cells become more flattened and more square formed on the surface of the culture dish. After 7 days, the cells starts becoming more fibroblast like. At this stage it is evident that the cells are dividing, spreading and are highly active. The most active cells are those from the cranial and jaw cartilage. Cells cultured in the medium supplemented with 10% Nu-Serum™ IV Culture Supplement and 1% ITS+™ Premix were more active and divide more rapidly, as compared to cells cultures in medium with the serum from the shark it-self.

After 10 days, the cultures that were seeded at the higher densities have reached confluence. Cells are now long and appear fibroblastitic.

Staining for the chondrocyte phenotypic characteristics initially indicates the chondrocyte phenotype (when cells are rounded), and as cells starts to flatten to become more fibroblastic on the substratum, thus the loss of this phenotypic behaviour.

The re-differentiation potential of the cultured chondrocytes is confirmed by the suspension cultures, in which cells in this liquid suspension had initially been cultured on the tissue culture dish. At the time of harvest, these cells had become differentiated and appeared fibroblastic. As the medium was removed from the suspension culture and plated out on the tissue culture dish, the cells again appeared rounded and staining for the cartilage specific components indicated the re-differentiation of cells to the chondrocyte phenotype. After 7 days in this culture, the cells again became more fibroblastic.

### Example 2

Extraction of anti-angiogenic fractions

When the growth period is finished the cells and the culture media are separated by centrifugation at 600 g for 10 minutes. Then the cells are trypsinised using trypsin EDTA solution for 5-10 minutes, which releases the cells from the T-flask. Then the cell solution is centrifuged at 600 g for 15 minutes at 4° C. and the supernatant is stored for later use. The cells in the retentate are disrupted and homogenised in distilled water and stirred for 2 hours at 4° C. to extract the water-soluble molecules obtaining an extract.

The extract is centrifuged at 13.600 g for 15 minutes at 4° C. and the supernatant and the culture media supernatant are filtered on a 24 μm Whatman filter to get rid of particles susceptible to affect the performance of an ultrafiltration column. The filtrated materials are then polled together and ultrafiltrated at 4° C. on a tangential flow filtration column having a porosity of 250 kDa. The supernatant is sterile filtered on 0.22 μm filter, stored in sterile bottles for further use.

The fraction may be further examined by one of the following tests.

### Example 3

In vitro assay of the extract

The activity of the fraction obtained may be assayed by any one of the following methods.

The hormone-dependent cancer cell lines ZR75-1 and MCF-7 cells are grown to a density of population of 20.000 cells/well on 24-well plaques or 15.000 cells/well on 6 well plaques. They are tested in the presence or absence of different concentrations of the extract (all experiments are performed in triplicates). The cells are grown in an incubator under a constantly humidified atmosphere containing 5% CO₂ at 37° C. for 3, 7, 17 days.

Culture media are withdrawn and replaced by fresh media every second day and the cell growth inhibition can be measured by direct cell counting of the cells or by measuring the total DNA content of a well.

In vivo assay of the extract

40 Days old female Spague-Dawley rats are adapted to their environment for 12 days. Then 20 mg MBA/ml corn oil (9,10-Dimethyl-1,2-Benzanthracene) was administered to the rats by gavage and three month after this treatment the rats having developed a mammary breast cancer have been selected.

These rats are distributed in groups where the rats in the treated groups are given a daily dose of increasing concentration of cartilage extract in 3 ml of water for eight weeks, while the control group received the same volume of pure water. The growth of inhibition can be measured by comparing the decrease of tumor diameter vs. a control.

CAM-assay

Another method for analyzing the activity of the extract is by the CAM-assay (chicken chorioallantoic membrane assay) which has been described by Sheu et al. (1998).

### Example 4

Toxicology

For evaluating the non-toxicity of the active molecules of the cartilage extract, the animals used in the above in vivo experiments were killed by decapitation and the following tissues were taken for analysis: liver, lung, kidneys, heart, brain, muscle and mammary gland. After they have been fixated for two days in Bouin fluid the fat was taken out of these tissues. After dehydration in ethanol, the fixated tissues were embedded in paraffin. Sections thereof were obtained and mounted on glass slides, coloured with haematoxlin and visualised under microscope.

### Example 5

### Cell counting

For following the growth of the chondrocytes the cells are trypsinised using trypsin EDTA for 5-10 minutes and counted using Trypan Blue viability staining using a hemocytometer. The count was adjusted to 5-10×10⁵ cells/ml.

After counting the cells is isolated by centrifugation at 600 g for 10 minutes and 4° C. and 10 ml of fresh media is added and the solution is incubated again at either 28° C., 21° C. or 9° C. where 5% CO₂ is added to the samples incubated at 28° C.

### References

Keuttner et al. (1977) No. 4,042,457

Spilburg et al. (1981) No. 4,243,582

Schinitsky (1984) No. 4,473,551

Kuettner et al. (1988) No. 4,746,729

Gabliks, J. (1988) No. 4,783,411

Dupont et al. (1997) No. 5,618,925

Dupont et al. (1999) No. 5,985,839

Langer, R et al. (1976), Science, 193:70-72.

Lee, A. and R. Langer (1983), Science, 221:1185-1187.

Folkman, J. and M. Klagsbrun (1987), Science, 235:442-446.

Oikawa et al (1990), Cancer Letters, 51:181-186

Marchalonis et al. (1990), Scand J, Immunol, 32(1)-13-20

McKinney et al. (1990), Cell Immunol., 127(2):506-513

Moore et al. (1993), Proc Natl Acad Sci USA, 90(4):1354-1358

Rosen and Woodhead, (1980), Med Hypotheses, 6(4):441-446

Coon and Cahn, (1966), Science, 153:1116

Cahn and Lasher, (1967), PNAS USA, 58:1131

Gospodarowicz and Mescher, (1977), J. Cell Physiology, 93:117

Benya et al., (1978), Cell, 15:1313

Brittberg et al., (1994), New Engl. J. Med., 331:889

Aulthourse et al., (1989), In Vitro Cell Dev. Biol., 25:659

Archer et al., (1990), J. Cell Sci., 97:361

Haanselmann et al., (1994), J. Cell Sci., 107:17

Bonaventure et al. (1994), Exp. Cell Res., 212:97

Kolettas et al., (1995), J. Cell Sci., 108:1991

Sheu et al., (1998), Anticancer Research, 18:4435-4442

Kuhn et al., (1978), In Vitro, 15(7): 537-544

No Author, (1999), Oncology, 13(1):82

1. A method of producing an anti-angiogenic, anti-inflammatoric, lysozomic and/or anticollagenolytic fraction and/or collagen and/or chondroitinsulphate from cultured chondrocytes, comprising
selecting a chondrocyte source having a sufficient amount of viable chondrocytes,
denuding the chondrocytes at least partially from extracellular matrix,
plating the denuded chondrocytes in high density in a culture medium,
separating the cultured chondrocytes from substantially all the culture medium, and
extracting a fraction comprising anti-angiogenic, anti-inflammatoric and/or anti-collagenolytic molecules and/or collagen and/or chondroitinsulphate from said cultured chondrocytes, obtaining the anti-angiogenic, anti-inflammatoric and/or anti-collagenolytic fraction and/or collagen and/or chondroitinsulphate.

2. The method according to claim 1, wherein the chondrocyte source is animal cartilage.

3. The method according to claim 2, wherein the chondrocyte source is elasmobranch cartilage.

4. The method according to claim 1, wherein the chondrocytes are elasmobranch chondrocytes.

5. The method according to claim 4, wherein the chondrocytes are ray or shark chondrocytes.

6. The method according to claim 1, wherein the chondrocyte source is a chondrocyte culture.

7. The method according to claim 1, wherein the chondrocytes are cultured at a temperature below 37° C.

8. The method according to claim 1, wherein the chondrocytes are cultured at a temperature below 29° C.

9. The method according to claim 1, wherein the chondrocytes are cultured in a medium containing serum.

10. The method according to claim 1, wherein the chondrocytes are cultured in a medium containing serum substitutions.

11. The method according to claim 1, wherein the chondrocytes are cultured in a monolayered culture.

12. The method according to claim 1, wherein the chondrocytes are cultured in a three-dimensional culture.

13. The method according to claim 12, wherein the chondrocytes are cultured at a scaffold or a mould.

14. The method according to claim 1, wherein the biochemical, physical and structural environments is approximately as in natural cartilage.

15. The method according to claim 1, wherein, during culturing, the chondrocytes are allowed to settle, before the culture medium is changed.

16. The method according to claim 15, wherein the chondrocytes are allowed to settle, preferably for at least 3 days, before the culture medium is changed.

17. The method according to claim 1, wherein the denuded chondrocytes are plated in a density of at least 5×10⁶ chondrocytes per ml.

18. The method according to claim 1, wherein the fraction is concentrated by ultrafiltration by use of a filter membrane having a porosity of 1600 kDa.

19. The method according to claim 1, wherein the fraction, comprising water soluble molecules, is combined with the culture medium, before obtaining said anti-angiogenic, anti-inflammatoric and/or anti-collagenolytic fraction.

20. The method according to claim 1, wherein the chondrocyte source has at least 50% viable chondrocytes.

21. The method according to claim 1, wherein the chondrocytes are cultured for a time sufficient for at least 10 cell division cycles.

22. The method according to claim 1, wherein the chondrocytes produce collagen.

23. The method according to claim 1, wherein the chondrocytes secrete components of cartilage.

24. The method according to claim 1, wherein the chondrocyte cells are immature and/or mature cells of the chondrocyte type and/or cells of a maturity in between immature and mature.

25. The method according to claim 1 wherein the chondrocyte cells are genetically manipulated.

26. The method according to claim 1, wherein the anti-angiogenic, anti-inflammatory and/or anti-collagenolytic fraction is further purified.

27. The method according to claim 17 wherein the anti-angiogenic, anti-inflammatoric and/or anti-collagenolytic fraction is freeze-dried.

28. A method of culturing elasmobranch chondrocytes comprising
selecting a chondrocyte source having a sufficient amount of viable chondrocytes,
denuding the chondrocytes from extracellular matrix,
plating the denuded chondrocytes in high density in a culture medium, and
culturing the chondrocytes at a temperature below 29° C.

29. An anti-angiogenic, anti-inflammatoric, lysozomic and/or anti-collagenolytic fraction and/or collagen and/or chondroifinsulphate obtainable by
selecting a chondrocyte source having a sufficient amount of viable chondrocytes,
denuding the chondrocytes from extracellular matrix,
plating the denuded chondrocytes in high density in a culture medium,
culturing the chondrocytes,
separating the cultured chondrocytes from the culture medium, and
extracting a fraction comprising anti-angiogenic, anti-inflammatoric, lysozomic and/or anti-collagenolytic molecules and/or collagen and/or chondroitinsulphate from said cultured chondrocytes, obtaining the anti-angiogenic, anti-inflammatoric, lysozomic and/or anti-collagenolytic fraction and/or collagen and/or chondroitinsulphate.

30. The fraction according to claim 29, said fraction being substantially free from molecules specific for fat tissue, muscle tissue and/or bone tissue.

31. The fraction according to claim 29 or 30, having origin from elasmobranch chondrocytes.

32. The fraction according to any of the claims 29-31, having molecules in the range of from 0 to 500 kDa.

33. An anti-angiogenic composition comprising an anti-angiogenic fraction from cultured chondrocytes as defined in any of the claims 29-32.

34. An anti-inflammatoric composition comprising an anti-inflammatoric fraction from cultured chondrocytes as defined in any of the claims 29-32.

35. An anti-collagenolytic composition comprising an anti-collagenolytic fraction from cultured chondrocytes as defined in any of the claims 29-32.

36. The composition according to claims 33-35 being formulated in a form suitable for oral administration.

37. The composition according to claims 33-35, wherein the composition is in the form of tablets, chewing gum, pellets, granules, powder, capsules, drops, sublingual wafers, or oral liquids.

38. The composition according to claims 33-35, being in the form of suppositories, injection preparations, cremes, pastes, or inhalation powders.

39. A method of treating and/or preventing a disease in an animal or human being, comprising administering to a patient a sufficient amount of an anti-angiogenic, anti-inflammatoric, lysozomic and/or anti-collagenolytic fraction and/or collagen and/or chondroitinsulphate as obtainable by the method defined in claim 1 or an anti-angiogenic, anti-inflammatoric, lysozomic and/or anti-collagenolytic composition as defined by claim 33-38.

40. The method according to claim 39, wherein the disease is a cancer disease.

41. The method according to claim 39, wherein the disease is psoriasis, or lupus.

42. The method according to claim 39, wherein the disease is an immunological disease, such as arthritis.

43. The method according to claim 39, wherein the disease is a diabetes related disease, such as diabetic renopati.

44. A method of treating and/or preventing a disease in an animal or human being, comprising planting a sufficient amount of cells within a joint of the animal or human body, in which the cells are obtained by the method defined in claim 1.

45. The method according to claim 44 or claim 39, wherein the disease is inflammatory joints.

46. The method according to claim 45, wherein the disease is rheumatoid arthritis.

47. Use of an anti-angiogenic, anti-inflammatoric, lysozomic and/or anti-collagenolytic fraction and/or collagen and/or chondroitinsulphate as obtainable by the method defined in claim 1 or an anti-angiogenic, anti-inflammatoric, lysozomic and/or anti-collagenolytic fraction 29-32 for the preparation of a composition for the treatment of a disease in a human or an animal.

48. The use according to claim 47, wherein the disease is a cancer disease.

49. The use according to claim 47, wherein the disease is psoriasis, or lupus.

50. The use according to claim 47, wherein the disease is an immunological disease, such as arthritis.

51. The use according to claim 47, wherein the disease is a diabetes related disease, such as diabetic renopati.

52. The use according to claim 47, wherein the disease is inflammatoric joints.

53. The use according to claim 52, wherein the disease is rheumatoid arthritis.

54. A chondrocyte culture as obtainable by
selecting a chondrocyte source having a sufficient amount of viable chondrocytes
denuding the chondrocytes from extracellular matrix
plating the denuded chondrocytes in high density in a culture medium, and
culturing the chondrocytes at a temperature below 29° C.

55. The culture according to claim 54, comprising elasmobranch chondrocytes.
